(19) United States
(12) Patent Application Publication
(10) Pub. No.: US 2012/0070487 A1
(43) Pub. Date: Mar. 22, 2012

(54) ALKYLSACCHARIDE COMPOSITIONS WITH NUTRACEUTICALS

(75) Inventor: Edward T. Maggio, San Diego, CA (US)

(73) Assignee: AEGIS THERAPEUTICS LLC, San Diego, CA (US)

(21) Appl. No.: 13/069,782

(22) Filed: Mar. 23, 2011

Related U.S. Application Data
(60) Provisional application No. 61/317,201, filed on Mar. 24, 2010.

(51) Int. Cl. A61K 9/127; A61K 38/02; A61K 33/06; A61K 31/714; A61P 3/02; A61K 31/12; A61K 31/7004; A61K 31/7048; A61K 47/42; A61K 47/26; A61K 31/05

(52) U.S. Cl. 424/450; 514/777; 514/5.5; 424/682; 514/52; 514/733; 514/679; 514/23; 514/27; 424/94.1

(57) ABSTRACT

The present invention provides nutraceutical compositions with enhanced oral bioavailability. The compositions of the present invention include one or more alkylsaccharides admixed with one or more nutraceutical.

### CROSS REFERENCE TO RELATED APPLICATIONS)

This application claims the benefit of priority under 35 U.S.C. §119(e) of U.S. Ser. No. 61/317,201, filed Mar. 24, 2010, the entire content of which is incorporated herein by reference.

### BACKGROUND OF THE INVENTION

1. Field of the Invention

The invention relates generally to nutraceutical compositions and more specifically to nutraceutical compositions including alkylsaccharides as well as methods for administering the compositions.

2. Background Information

Certain foods or food products have been determined to provide health benefits in prevention and treatment of disease and have been designated as nutraceuticals. These products can include dietary supplements, isolated nutrients and herbal products by way of example. They may also include vitamins, minerals, and amino acids. A limitation in the uptake of many of these nutraceuticals relates to poor absorption in the stomach or intestines, also referred to as poor bioavailability. In some instances, poor absorption may also be exacerbated by disease states such as celiac disease.

### SUMMARY OF THE INVENTION

It has been discovered that certain well tolerated and generally recognized as safe alkylsaccharides can be admixed with various nutraceuticals, and when administered orally to a human or animal subject, significantly increase the bioavailability of nutraceutical components. As such, the present invention relates to formulations of alkylsaccharides admixed with nutraceuticals. Accordingly, the present invention provides a composition including a nutraceutical and an alkylsaccharide, in a pharmaceutical carrier. In various embodiments, the alkylsaccharide has an alkyl chain including between 10 to 16 carbons. In various embodiments, the alkylsaccharide may be sucrose cocoate, n-dodecyl-beta-D-maltoside, n-tetradecyl-beta-D-maltoside, sucrose laurate, sucrose myristate, sucrose palmitate, tridecyl-beta-D-maltoside, sucrose mono-dodecanoate, sucrose mono-tridecanoate, and sucrose mono-tetradecanoate.

In another aspect, the present invention provides a method of administering a nutraceutical to a subject. The method administering a composition including a nutraceutical admixed and an alkylsaccharide to the subject, thereby administering the nutraceutical to the subject. In various embodiments, the alkylsaccharide has an alkyl chain including between 10 to 16 carbons. In various embodiments, the alkylsaccharide may be cocoate, n-dodecyl-beta-D-maltoside, n-tetradecyl-beta-D-maltoside, sucrose laurate, sucrose myristate, sucrose palmitate, tridecyl-beta-D-maltoside, sucrose mono-dodecanoate, sucrose mono-tridecanoate, and sucrose mono-tetradecanoate. In various embodiments, the one or more insulin analogs is administered via the oral, buccal, nasal, nasolacrimal, inhalation, pulmonary, transdermal or CSF delivery route.

### DETAILED DESCRIPTION OF THE INVENTION

The present invention describes compositions comprising an alkylsaccharide having certain structural characteristics in combination with a nutraceutical.

Before the present composition and method are described, it is to be understood that this invention is not limited to the particular composition, method, and experimental condition described. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, “alkylsaccharide” refers to any sugar joined by a linkage to any hydrophobic alkyl, as is known in the art. Preferably the alkylsaccharide is nonionic as well as nontoxic and considered Generally Recognized As Safe, for food applications, sometimes referred to as a GRAS substance. Alkylsaccharides are available from a number of commercial sources and may be natural or synthesized by known procedures, such as chemically or enzymatically.

In various aspects, alkylsaccharides of the present invention may include, but not limited to: alkylglycosides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers.

As described above, the hydrophobic alkyl can thus be chosen of any desired size, depending on the hydrophobicity desired and the hydrophilicity of the saccharide moiety. For example, one preferred range of alkyl chains is from about 10 to about 24 carbon atoms. An even more preferred range is from about 10 to about 16 or about 14 carbon atoms. Similarly, some preferred glycosides include maltose, sucrose, and glucose linked by glycosidic linkage to an alkyl chain of 9, 10, 12, 13, 14, 16, 18, 20, 22, or 24 carbon atoms, for example, nonyl-, decyl-, dodecyl-, tridecyl, and tetradecyl sucroside, glucoside, maltoside, and the like. These compositions are nontoxic, since they are degraded to an alcohol or fatty acid and an oligosaccharide, and amphipathic. Additionally, the linkage between the hydrophobic alkyl group and the hydrophilic saccharide can include, among other possibilities, a glycosidic, thioglycosidic, amide, ureide, or ester linkage.

In sugar chemistry, an anomer is either of a pair of cyclic stereoisomers (designated α or β) of a sugar or glycoside, differing only in configuration at the hemiacetal (or hemiketal) carbon, also called the anomeric carbon or reducing carbon. If the structure is analogous to one with the hydroxyl group on the anomeric carbon in the axial position of glucose, then the sugar is an alpha anomer. If, however, that hydroxyl is equatorial, the sugar is a beta anomer. For example, dodecyl β-D-maltoside and dodecyl α-D-maltoside are two cyclic forms of dodecyl maltoside and are anomers. The two different anomers are two distinct chemical structures, and thus have different physical and chemical properties. In one embodiment of the invention, the alkylsaccharide for use with the present invention is a β anomer. In an exemplary aspect, the alkylsaccharide is a β anomer of dodecyl maltoside, tridecyl maltoside or tetradecyl maltoside.

In one embodiment of the present invention, the alkylsaccharide used is a substantially pure alkylsaccharide. As used herein a “substantially pure” alkylsaccharide refers to one anomeric form of the alkylsaccharide (either the α or β anomeric forms) with less than about 2% of the other anomeric form, preferably less than about 1.5% of the other anomeric form, and more preferably less than about 1% of the other anomeric form. In one aspect, a substantially pure alkylsaccharide contains greater than 98% of either the α or β anomer. In another aspect, a substantially pure alkylsaccharide contains greater than 99% of either the α or β anomer. In another aspect, a substantially pure alkylsaccharide contains greater than 99.5% of either the α or β anomer. In another aspect, a substantially pure alkylsaccharide contains greater than 99.9% of either the α or β anomer.

Some exemplary glycosides include maltose, sucrose, and glucose linked by glycosidic linkage to an alkyl chain of 9, 10, 12, 14 or 16 carbon atoms, i.e., nonyl-, decyl-, dodecyl-, tetradecyl- and hexadecyl sucroside, glucoside, and maltoside. As discussed above, these compositions are nontoxic, since they are degraded to long chain alcohols or corresponding long chain fatty acids which are common and normal dietary constituents, and an oligosaccharide. Particular examples include, but are not limited to sucrose cocoate, n-Dodecyl-4-O-α-D-glucopyranosyl-β-D-glucopyranoside (dodecyl-β-D-maltoside) or n-tetradecyl-4-O-α-D-glucopyranosyl-β-D-glucopyranoside (tetradecyl-β-D-maltoside), sucrose laurate, sucrose myristate, sucrose palmitate and mixtures thereof. It is also beneficial if the alkylglycoside chosen is metabolized or eliminated by the body and if this metabolism or elimination is done in a manner that will not be harmfully toxic. Additional saccharides useful in the present invention owing to their safety upon being metabolized in the body include glucose, maltotriose, maltotetraose, and trehalose.

“Antioxidant” as used herein refers to a molecule capable of slowing or preventing the oxidation of other molecules.

“Binding agent” as used herein refers to a compound that binds together various components for improving yield during production of a powder form.

“Emulsion” as used herein refers to a mixture of two immiscible liquids, a water soluble liquid and an oil soluble liquid.

“Flavonoids” as used herein refers to a class of secondary plant metabolites having a three-ringed flavone backbone, known for their antioxidant activity.

“Nutraceutical” as used herein refers to a composition comprising a combination of extracts claimed to have a physiological benefit on human health and/or reduce the risk of chronic disease in humans.

“Phenols” or “phenolics” as used herein refers to chemical compounds consisting of a hydroxyl group (—OH) bonded directly to an aromatic hydrocarbon group.

“Phytochemical” or “phytonutrient” as used herein refers to plant-derived chemical compounds under scientific research for their potential health promoting properties.

“Polyphenols” as used herein refers to a group of chemical substances characterized by the presence of more than one phenol unit or building block per molecule.

“Saponin” as used herein refers to amphipathic glycosides composed of one or more hydrophilic glycoside moieties combined with a lipophilic triterpene derivative.

Examples of nutraceuticals include but are not limited to: nutritional peptides, such as plant or animal meal, resveratrol, calcium salts including calcium carbonate, calcium citrate and calcium ascorbate, Vitamin B12, cranberry anthocyanins, curcumin, tagatose, berberine, red yeast rice, policosanols, phosphatidylserine, Ginkgo biloba, vitamin E, folate, pyridoxine, S-adenosyl-1-methionine, sweet lupin proteins, quercetin, inositol hexaphosphate, quercetin., beta-glucans of Agaricus brasiliensis, phytochemicals including polyphenolic compounds, squalene, alpha-tocopherol, carotenoid, plant extracts such as Capparis spinosa, Olea europaea, Panax Ginseng and Ribes nigrum, chondroitin sulphate, glucosamine, colostrinin, anthocyanin, cosequin, deacetylescins Ia, IIa, Ib, and IIb as well as two types of desacylescins I and II, gamma-tocotrienol, acarbose, soluble fiber—most notably glucomannan, chlorogenic acid, chromium picolinate, phytanic acid, taurine, policosanol; 9-cis-beta-carotene vitamin D, genistein, sesamin, mushroom beta-glucans, flavan-3-ol (flavanol), glucosinolates, phytochemicals of broccoli, sesame lignans, boswellia serrata, and the n-3 polyunsaturated fatty acids, alpha-linolenate or eicosapentaenoic acid, beta carotene, anthocyanins among others, stilbenoids, phenols, phytoalexin, flavonids, saponins, phytonutrients, and polyphenols. Additional nutraceuticals intended for use in the present invention are further described below.

Generally, the major nutrient constituents in naturally-derived nutraceuticals are antioxidants, which are known to be efficacious in the treatment and prevention of a wide range of chronic illnesses. Aquatic animal oil, in particular krill oil or fish oil such as cod liver oil shark liver oil are a rich source of antioxidants such as omega-3 fatty acids and squalene. Examples of phyto-antioxidants found in vegetables and herbs are pigments such as carotenoids and phenolics or more accurately described as polyphenols such as flavonoids, saponins, tocopherols and tocotrienols. Polyphenol antioxidants are generally believed to be instrumental in combating oxidative stress in humans, a process associated with some neurodegenerative diseases and some cardiovascular diseases.

Flavonoids are well known water soluble antioxidants and have relatively low toxicity in comparison to other phytochemical compounds. Flavonoids may be divided into six subclasses e.g. anthocyanidins, flavanols, flavanones, flavonols, flavones and isoflavones. Examples of common dietary flavonoids are resveratrol (red wine), catechin (tea), epicatechin (cocoa), hesperidin (citrus fruits), genistine and daidzein (soybean) and quercetin (capers).

Saponins are amphipathic glycosides that dissolve in water to form a stable soapy froth. As a natural surfactant, they are often used as emulsifiers and are excellent detergents. Use of saponins in nutraceuticals for controlling cholesterol-related illnesses has been notably successful. Saponin acts to lower blood cholesterol levels by binding cholesterol and preventing its re- absorption into the blood circulatory system, when consumed. Natural saponins have been known to alter permeability of cells (promote absorption of medicine), and are capable of altering the surface tension of water. The antibacterial, antiviral, antifungal and detoxification properties of saponin have also been documented in numerous scientific studies.

Other examples of phytonutrients are trace minerals such as potassium, calcium and magnesium. These minerals are documented to be efficacious in preventing and managing certain mineral-deficiency mitigated disorders.

Carotenoids (vitamin A) are organic pigments naturally occurring in the chromoplasts of plants. Along with tocopherols and tocotrienols (vitamin E), carotenoids are examples of phytonutrients that are natural oil-soluble antioxidants believed to play a significant role in the prevention of cancer, cellular aging and/or treatment of atherosclerosis, arthritis and Alzheimer's disease, and are available in significant amounts in some edible vegetable oils such as red palm oil, wheat germ oil, coconut oil, corn oil, soya bean oil, olive oil, sunflower oil, rice bran oil or grape seed oil.

Compositions comprising an alkylsaccharide and a nutraceutical may be presented in a number of forms such as solutions, suspensions, dried powders, encapsulated or tableted dry mixtures, oil enclosed gel capsules, and the like with or without other formulation components sometimes called excipients such as bulking agents, tableting agents, dissolution agents, wetting agents, lubricants, colors, flavors disintegrants, coatings, binders, antioxidants, sweeteners, and the like, as are commonly used in the pharmaceutical industry.

Examples include magnesium stearate, sorbitol, mannitol, xylitol,hydroxyl cellulose, lactose, starch, sugars, maltitol, polyethylene glycol, cellulose, gelatin, polyvinylpyrrolidone, carboxymethylcellulose, silicon dioxide, talc, magnesium or sodium carbonate, acelfame, aspartame, cyclamate, saccharin, thaumatin glycyrrhizin and many others well known to those skilled in the art of pharmaceutical formulation for oral administration.

The pharmaceutical compositions described herein are formulated for various routes of administration, such as, oral, buccal, nasal, nasolacrimal, inhalation, pulmonary, transdermal or CSF administration. As such, in addition to nutraceuticals and alkylsaccharides, the compositions may further include one or more of an aggregation inhibitory agent; a charge-modifying agent; a pH control agent; a degradative enzyme inhibitory agent; a mucolytic or mucus clearing agent; a ciliostatic agent; or a membrane penetration-enhancing agent.

Examples of membrane penetration-enhancing agents include cyclodextrins, such as methyl-beta-cyclodextrin; alkylglycosides, such as dodecylmaltoside and tetradecylmaltoside; an aggregation inhibitory agent; a charge-modifying agent; a pH control agent; a degradative enzyme inhibitory agent; a mucolytic or mucus clearing agent; a ciliostatic agent; a membrane penetration-enhancing agent selected from: (i) a cyclodextrin such as methyl-beta-cyclodextrin; an alkylglycoside or other surfactant; (ii) a bile salt; (ii) a phospholipid additive, mixed micelle, liposome, or carrier; (iii) an alcohol; (iv) an enamine; (v) an NO donor compound; (vi) a long-chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); a modulatory agent of epithelial junction physiology; a vasodilator agent; a selective transport-enhancing agent; and a stabilizing delivery vehicle, carrier, mucoadhesive, support or complex-forming species with which the compound is effectively combined, associated, contained, encapsulated or bound resulting in stabilization of the compound for enhanced delivery, wherein the formulation of the compound with the delivery-enhancing agents provides for increased bioavailability of the compound in a blood plasma of a subject.

Examples of preservatives that may be used in the compositions of the present invention, include, but are not limited to preservatives such as ethylene diamine tetraacetic acid (EDTA), sodium azide, p-hydroxybenzoate and its analogs, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, chlorobutanol, m-cresol and alkyglycosides such as dodecyl maltoside.

In various embodiments, the compositions described herein may further include one or more excipients including stabilizers, surfactants, antimicrobial agents, osmolarity adjusting agents such as mannitol, sorbitol or sodium chloride.

Further, the compositions described herein may be buffered to have a pH of about 4 to 8, 4.5 to 7.5, 4.5 to 6.5, or 5 to 6.

The present invention also provides a method of administering a nutraceutical to a subject. The method includes administering a composition compromising a nutraceutical admixed with an alkylsaccharide to the subject, thereby administering the nutraceutical to the subject.

As described herein, the compositions of the invention may be formulated for, and delivered via any suitable administration route, including, for example, oral, buccal, nasal, nasolacrimal, inhalation, pulmonary, transdermal or CSF delivery routes. The terms “administration” or “administering” as used herein are defined to include the act of providing a pharmaceutical composition of the invention to a subject in need of treatment. While the compositions described herein may be suitable for administration via any well known route, an exemplary administration route is orally. Accordingly, in an exemplary aspect, the compositions of the present invention, are formulated into acceptable forms suitable for oral administration, such as tablets, capsules or pills.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, and the like, and primates (including monkeys, chimpanzees, orangutans and gorillas) are included within the definition of subject.

The following examples are intended to illustrate but not limit the invention.

### EXAMPLE 1

### Oral Adsorption of Peptides

This example shows the enteral uptake of the seven amino acid peptide “Ser-Cys-Ser-D-Leu-Pro-Gln-Thr” hereafter designated [D-Leu-4]OB3 formulated with 0.3% n-tetradecyl-beta-D-maltoside upon oral administration to six-week old male Swiss Webster mice (Taconic Farms, Germantown, N.Y.).

Doses of 1 mg each were also administered to mice by the IP, SC and IM routes in solutions containing 0.18% tetradecyl beta-D-maltoside and compared to administration by oral gavage in a solution containing 0.3% (3 mg/mL wt/vol) of tetradecyl-beta-D-maltoside.

Six week-old male Swiss Webster mice weighing approximately 30 gm were maintained at a constant temperature (24 C) with lights on from 07:00 to 19:00 h, and allowed food and water ad libitum until used for uptake studies. Mouse [D-Leu-4]OB3 was prepared commercially as a C-terminal amide by Bachem (Torrance, Calif., USA). For sc, im, and ip delivery, the peptide was dissolved in sterile phosphate buffered saline (PBS, pH 7.2) at a concentration of 1 mg/200 μL as described previously containing 0.18% tetradecyl-beta-D-maltoside. For oral gavage [D-Leu-4]OB3 was dissolved in 0.3% dodecyl-beta-D-maltoside reconstituted in PBS (pH 7.2) at a concentration of 1 mg/200 μL. At time zero (0), a single 200 μL, sc, im, ip or oral dose of mouse [D-Leu-4]OB3 was given to each of six mice per time point. Following peptide administration, the mice were transferred to separate cages for the designated time period.

Five, 10, 20, 40, 60, or 120 minutes after peptide delivery, the mice were anesthetized with isoflurane (5%) and exsanguinated by cardiac puncture. The blood was collected in sterile nonheparinized plastic centrifuge tubes and allowed to stand at room temperature for 1 h. The clotted blood was rimmed from the walls of the tubes with sterile wooden applicator sticks. Individual serum samples were prepared by centrifugation for 30 min at 2600×g in an Eppendorf™ 5702R, A-4-38 rotor (Eppendorf North America, Westbury, N.Y., USA), and stored frozen until assayed for mouse [D-Leu-4]OB3 content by competitive ELISA.

The [D-Leu-4]OB3 competitive ELISA assay was carried out as follows. 96-well polystyrene plates (Pierce Biotechnology, Inc., Rockford, Ill., USA) were coated with 100 μL of a 5 ug/ml solution of BSA-conjugated mouse [D-Leu-4]OB3 (QED Bioscience, San Diego, Calif.) in carbonate-bicarbonate buffer (pH 9.4). The coated plates were incubated overnight at 4 C. Unoccupied sites were blocked with 200 μL StartingBlock™ in PBS (Pierce Biotechnology Inc., Rockford, Ill., USA) for 2 h at room temperature. Mouse [D-Leu-4]OB3 standards ranging from 5 to 10,000 ng/ml are prepared in PBS (pH 7.2). In a separate incubation, 100 μL of mouse [D-Leu-4]OB3 primary antibody raised in New Zealand White rabbits (QED Bioscience, San Diego, Calif.) and diluted to 1:5000 in StartingBlock™, or 100 μL of the primary antibody+50 μL of each standard or serum sample are added to 500 μL microcentrifuge tubes and incubated for 1 h at 37 C. At the end of the incubation period, 100 μK of each antibody-bound standard or sample were added to the wells and incubated for 1 h at room temperature. HRP-conjugated goat-anti-rabbit IgG (Pierce Biotechnology Inc, Rockford, Ill., USA) was used as the secondary antibody. 100 μL was added to each well and incubated for 1 h at room temperature. At the end of the incubation period, 100 μL of ABTS substrate (Pierce Biotechnology Inc, Rockford, Ill., USA) was added to each well and incubated for 30 minutes on a rotary rocker. Color development was stopped with 1% SDS. Absorbance was read at 405 nm with a Molecular Devices microplate reader (MDS Sciex, Concord, Ontario Canada). Each sample was assayed in triplicate. Intra-assay and inter-assay coefficients of variation are 0.04% and 0.2%, respectively. The relative bioavailability was determined by plotting the serum concentrations of mouse [D-Leu-4]OB3 vs. time following ip, sc, or im delivery using the graphics program SigmaPlot™ 8.0 (SPSS Science, Chicago, Ill., USA). The area under each curve (AUC) was calculated with a function of this program. The relative oral bioavailability compared to the three injection modes is presented below in Table 3. For example, oral gavage achieved 47.3% oral bioavailability compared to subcutaneous injection.

TABLE 1

| | | | Relative Oral |
|---|---|---|---|
| | | | Bioavailability Compared |
| | | | to Three Injection Modes |
| Alkylsaccharide | Delivery | AUC | (% of AUC achieved by |
| (%) | Route | (ng/ml/min) | each respective injection) |
| 0.3% dodecyl-beta- | Oral | 559,330 | — |
| D-maltoside | gavage | | |
| 0.18% tetradecyl- | IP | 1,072,270 | 52.2% of IP AUC |
| beta-D-maltoside | | | |
| 0.18% tetradecyl- | SC | 1,182,498 | 47.3% of SC AUC |
| beta-D-maltoside | | | |
| 0.18% tetradecyl- | IM | 1,481,060 | 37.8% of IM AUC |
| beta-D-maltoside | | | |

### EXAMPLE 2

### Increased Oral Absorption of Reseratrol

Resveratrol was obtained from RevGenetics, Miami, Fla., as 300 mg gel capsules. Capsules were opened by carefully separating the two halves. The contents were combined and admixed with 5% w/w food grade sucrose laurate alkylsaccharide (Ryoto Sugar Ester M-1216, Mitsubishi-Kagaku Tokyo, Japan). Gelatin capsules were filled with the admixture and administered to volunteers. Blood samples are collected and resveratrol levels are measured by the method of Soleas et al. (Methods Enzymol 335: 130-45 (2001)). The resveratrol blood levels obtained following oral administration with sucrose laurate are at least 20% higher than those obtained in the absence of sucrose laurate.

### EXAMPLE 3

### Increased Oral Absorption of Calcium

Calcium citrate is obtained from General Nutrition Corp. as 1000 mg tablets. The tablets are ground in a mortar and pestle until a fine uniform powder is created. The powder is admixed with 3% dodecyl maltoside, pharmaceutical grade (Inalco Chemical Co., Milan Italy) and filled into gelatin capsules, size #000-25 mm length (Capsuline, Pompano Beach, Fla.) and administered to volunteers. Blood samples are collected and calcium levels are measured by commercial clinical laboratory. The calcium blood levels obtained following oral administration with dodecyl maltoside are seen to be at least 20% higher than those obtained in the absence of dodecyl maltoside. Similar results are obtained with 3% w/w sucrose laurate or 5% w/w concentration of sucrose cocoate.

Although the invention has been described with reference to the above example, it will be understood that modifications and variations are encompassed within the spirit and scope of the invention. Accordingly, the invention is limited only by the following claims.

What is claimed is:

1. A composition comprising:
a) a nutraceutical; and
b) an alkylsaccharide, in a pharmaceutical carrier.

2. The composition of claim 1, wherein the alkylsaccharide has an alkyl chain including between 10 to 16 carbons.

3. The composition of claim 1, wherein the alkylsaccharide is selected from the group consisting of sucrose cocoate, n-dodecyl-beta-D-maltoside, n-tetradecyl-beta-D-maltoside, sucrose laurate, sucrose myristate, sucrose palmitate, tridecyl-beta-D-maltoside, sucrose mono-dodecanoate, sucrose mono-tridecanoate, and sucrose mono-tetradecanoate.

4. The composition of claim 1, wherein the nutraceutical is selected from the group consisting of a nutritional peptide, resveratrol, calcium, Vitamin B12, cranberry anthocyanins, curcumin, and tagatose.

5. The composition of claim 1, further comprising a buffering agent.

6. The composition of claim 1, further comprising a mucosal delivery-enhancing agent selected from the group consisting of an aggregation inhibitory agent, a charge-modifying agent, a pH control agent, a degradative enzyme inhibitory agent, a mucolytic or mucus clearing agent, a chitosan, and a ciliostatic agent.

7. The composition of claim 1, further comprising benzalkonium chloride or chloroethanol.

8. The composition of claim 1, further comprising an agent selected from the group consisting of a surfactant, a bile salt, a phospholipid additive, a mixed micelle, a liposome, a carrier, an alcohol, an enamine, a nitric oxide donor compound, a long-chain amphipathic molecule, a small hydrophobic penetration enhancer, a sodium or a salicylic acid derivative, a glycerol ester of acetoacetic acid, a cyclodextrin or beta-cyclodextrin derivative, a medium-chain fatty acid, a chelating agent, an amino acid or salt thereof, an N-acetylamino acid or salt thereof, an enzyme degradative to a selected membrane component and any combination thereof.

9. The composition of claim 1, further comprising a modulatory agent of epithelial junction physiology.

10. The composition of claim 1, further comprising a vasodilator agent.

11. The composition of claim 1, further comprising a selective transport-enhancing agent.

12. The composition of claim 1, further comprising at least one excipient selected from the group consisting of bulking agents, tableting agents, dissolution agents, wetting agents, lubricants, colors, flavors, disintegrants, coatings, binders, antioxidants, and sweeteners.

13. The composition of claim 1, wherein the composition is formulated as a degradable capsule, tablet or pill.

14. A method of administering a nutraceutical to a subject, comprising administering a composition comprising a nutraceutical admixed and an alkylsaccharide to the subject, thereby administering the nutraceutical to the subject.

15. The method of claim 14, wherein the nutraceutical is administered via the oral, buccal, nasal, nasolacrimal, inhalation, pulmonary, transdermal or CSF delivery route.

16. The method of claim 14, wherein the alkylsaccharide has an alkyl chain including between 10 to 16 carbons.

17. The method of claim 14, wherein the alkylsaccharide is selected from the group consisting of sucrose cocoate, n-dodecyl-beta-D-maltoside, n-tetradecyl-beta-D-maltoside, sucrose laurate, sucrose myristate, sucrose palmitate, tridecyl-beta-D-maltoside, sucrose mono-dodecanoate, sucrose mono-tridecanoate, and sucrose mono-tetradecanoate.

18. The method of claim 14, wherein the nutraceutical is selected from the group consisting of a nutritional peptide, resveratrol, calcium, Vitamin B12, cranberry anthocyanins, curcumin, and tagatose.

19. The method of claim 14, wherein the composition further comprising a buffering agent.

20. The method of claim 14, wherein the composition further comprising a mucosal delivery-enhancing agent selected from the group consisting of an aggregation inhibitory agent, a charge-modifying agent, a pH control agent, a degradative enzyme inhibitory agent, a mucolytic or mucus clearing agent, a chitosan, and a ciliostatic agent.

21. The method of claim 14, wherein the composition further comprising benzalkonium chloride or chloroethanol.

22. The method of claim 14, wherein the composition further comprising an agent selected from the group consisting of a surfactant, a bile salt, a phospholipid additive, a mixed micelle, a liposome, a carrier, an alcohol, an enamine, a nitric oxide donor compound, a long-chain amphipathic molecule, a small hydrophobic penetration enhancer, a sodium or a salicylic acid derivative, a glycerol ester of acetoacetic acid, a cyclodextrin or beta-cyclodextrin derivative, a medium-chain fatty acid, a chelating agent, an amino acid or salt thereof, an N-acetylamino acid or salt thereof, an enzyme degradative to a selected membrane component and any combination thereof.

23. The method of claim 14, wherein the composition further comprising at least one excipient selected from the group consisting of bulking agents, tableting agents, dissolution agents, wetting agents, lubricants, colors, flavors, disintegrants, coatings, binders, antioxidants, and sweeteners.

24. The method of claim 14, wherein the composition is formulated as a degradable capsule, tablet or pill.
